SEQUENCE LISTING (2 sequences)

SEQ ID NO: 1 - DNA, 23 bp, Artificial Sequence
  Bap1F1 primer
ctcaatattccaccctgcgtctg

SEQ ID NO: 2 - DNA, 22 bp, Artificial Sequence
  Bap1R1 primer
ggcaggtggcccctctactcta

(19) United States
(12) Patent Application Publication
(10) Pub. No.: US 2022/0023293 A1
(43) Pub. Date: Jan. 27, 2022

(54) EZH2- FGFR INHIBITION IN CANCER

(71) Applicant: Stichting Het Nederlands Kanker Instituut-Antoni van Leeuwenhoek Ziekenhuis, Amsterdam (NL)

(72) Inventors: Antonius Jozef Maria BERNS, Amsterdam (NL); Jitendra BADHAI, Amsterdam (NL); Maarten Matthijs Sharif VAN LOHUIZEN, Amsterdam (NL)

(21) Appl. No.: 17/297,038

(22) PCT Filed: Dec. 3, 2019
(86) PCT No.: PCT/NL2019/050799

§ 371 Date: May 26, 2021

(30) Foreign Application Priority Data
Dec. 3, 2018 (EP) 18209921.8

(51) Int. Cl. A61K 31/496; A61K 31/502; A61K 31/5377; A01K 67/027

(52) U.S. Cl. CPC A61K 31/496; A61K 31/502; A61K 31/5377; A01K 2217/206; A01K 2227/105; A01K 2267/0331; A01K 2217/075; A01K 67/0276

(57) ABSTRACT

The invention relates to a combination of a FGFR inhibitor and an EZH2 inhibitor for use in a method of treating a patient suffering from a BRCA1-associated protein 1 (BAP1) negative cancer. The invention further relates to a pharmaceutical preparation, comprising a FGFR inhibitor and an EZH2 inhibitor, to the use of said pharmaceutical preparation in a method of treating BAP1-negative cancer, to methods of identifying a patient with cancer, who is eligible for treatment with a combination of a FGFR inhibitor and an EZH2 inhibitor, and to a mouse model for mesothelioma.

### FIELD

The invention relates to methods for treatment of cancer using a combination of inhibitors of EZH2 and FGFR1-3. Said cancer is mesothelioma, especially BAP1-negative mesothelioma.

### 1 BACKGROUND OF THE INVENTION

Malignant mesothelioma(MM) is a highly aggressive tumor of serosal surfaces. The majority of cases are linked to asbestos exposure and takes as long as 30-50 years for tumors to arise [McDonald and McDonald, 1996. Eur Respir J 9: 1932-42; Robinson et al., 2005. Lancet 366: 397-408]. The genomic landscape of MM shows frequent inactivation of CDKN2AB locus that encodes for p16INK4A, p15INK4B, and p14ARF cell cycle inhibitor proteins and Neurofibromatosis Type 2 (NF2) with well-established tumor suppressor function [Sekido et al., 1995. Cancer Res 55: 1227-31; Bianchi, et al., 1995. Proc Natl Acad Sci USA 92: 10854-8; Cheng et al., 1994. Cancer Res 54: 5547-51]. Recently, the BRCA1 associated protein 1 (BAP1) gene was found to be mutated, deleted or epigenetically silenced in 60-70% of human MM [Bott et al., 2011. Nat Genet 43: 668-72; Nasu et al., 2015. J Thorac Oncol 10: 565-76; Cigognetti et al., 2015. Mod Pathol 28: 1043-57; Testa et al., 2011. Nat Genet 43: 1022-5; Lo Iacono et al., 2015. J Thorac Oncol 10: 492-9].

MM can be subdivided into three histological subtypes: epithelioid, sarcomatoid, and biphasic [McCaughey, 1965. Ann N Y Acad Sci 132: 603-13]. Formal diagnosis of mesothelioma subtypes requires histological specimen to assess both tumor morphology in combination with immuno-histochemical markers [Ordonez, 2005. Arch Pathol Lab Med 129: 1407-12].

Inflammation is a hallmark of asbestos deposition in tissue and contributes to carcinogenesis. The vast majority of cases of MM is caused by asbestos exposure; therefore, inflammation and the immune microenvironment are believed to play a pivotal role in mesothelioma pathogenesis [Carbone et al., 2012. J Cell Physiol 227: 44-58]. Many different oncogenic signaling pathways have been implicated in mesothelioma pathogenesis. The most prominent pathways reported are PI3K-mTOR-AKT and MAPK pathways [Perrone et al., 2010. Eur J Cancer 46: 2837-48; Menges et al., 2010. Genes Cancer 1: 493-505; Brevet et al., 2011. J Thorac Oncol 6: 864-74; Suzuki et al., 2009. Mol Med Rep 2: 181-8; Ou et al., 2011. Oncogene 30: 1643-52; Altomare et al., 2005. Oncogene 24: 6080-9; Jagadeeswaran et al., 2006. Cancer Res 66: 352-61]. Additionally, recent literature suggests many epigenetic modulators such as KDM6A, SETD2, SETDB6, and EZH2 that may play a critical role in MM development [Vandermeers et al., 2013. Lung Cancer 81: 311-8; Manente et al., 2016. Epigenomics 8: 1227-38; LaFave et al., 2015. Nat Med 21: 1344-1349].

The only front-line treatment approved for mesothelioma is cisplatin in combination with either pemetrexed or raltitrexed yielding a modest survival benefit [Vogelzang et al., 2003. J Clin Oncol 21: 2636-44]. Carboplatin is an acceptable alternative to cisplatin in first line but there is currently no standard second line treatment. To date, no targeted therapies or immunotherapy have been approved for mesothelioma despite many clinical studies [Pinton et al., 2013. Expert Opin Investig Drugs 22: 1255-63; Ladanyi et al., 2012. Clin Cancer Res 18: 4485-90].

Simultaneous inactivation of Nf2 and Trp53 in thorax of mouse give rise to mesothelioma [Jongsma et al., 2008. Cancer Cell 13: 261-71]. In addition, including loss of Cdkn2a further accelerated tumor development [Jongsma et al., 2008. Cancer Cell 13: 261-71]. However, the frequency of TP53 mutations is relatively modest in human MM [Papp et al., 2001. Int J Oncol 18: 425-33; Metcalf et al., 1992. Cancer Res 52: 2610-5]. Recently, BAP1 mutations and deletions have been reported to occur in more than half of the mesothelioma patients. BAP1 belongs to the ubiquitin C-terminal hydrolase subfamily of deubiquitinating enzymes that biochemically deubiquitinate histone H2A [Scheuermann et al., 2010. Nature 465: 243-7]. We have explored whether Bap1 inactivation alone or in combination with the loss of other tumor suppressors can induce mesothelioma in mice. Recently, two studies reported mesothelioma induction in Bap1 deleted and mutated mice by providing asbestos fibers exposure [Napolitano et al., 2015. Oncogene volume 35: 1996-2002; Xu et al., 2014. Cancer Res 74: 4388-97; Kadariya et al., 2016. Cancer Res 76: 2836-44.]. They showed that Bap1+/−mice needed induction with asbestos to give rise to mesothelioma after a very long latency period [Napolitano et al., 2015. Oncogene volume 35: 1996-2002; Xu et al., 2014. Cancer Res 74: 4388-97]. Several research groups have attempted to make mouse models of MM in past. Davis et al. first modeled asbestos induced mesothelioma in mouse [Davis et al., 1992. Int J Cancer 52: 881-6]. Subsequently, Marsella et al. modeled crocidolite induced mesothelioma in Trp53 deficient mice [Marsella et al., 1997. Environ Health Perspect 105 Suppl 5: 1069-72] and Fleury-Feith et al in Nf2 hemizygous mice[Fleury-Feith et al., 2003. Oncogene 22: 3799-805]. Thereafter, Joseph Testa's group reported induction of mesothelioma in Nf2 deficient mice in 2005 [Bianchi, et al., 1995. Proc Natl Acad Sci USA 92: 10854-8; Altomare et al., 2005. Cancer Res 65: 8090-8095] and Cdkn2ab deficient mice in 2011 [Altomare et al., 2011. Plos One 6: e18828].

Most of the current mouse MM models do not harbor a combination of the predominant primary genetic lesions found in human MM and, consequently, a detailed insight into their contribution to the tumor phenotype and their involvement in specific oncogenic signaling pathways is lacking. Furthermore, the current models require a considerable time to develop mesothelioma and are therefore less suitable as a therapeutic intervention model. Therefore, there is need for a faster model of MM that harbors the frequently occurring genetic lesions in human MM and that accurately recapitulates human disease.

### BRIEF DESCRIPTION OF THE INVENTION

Compound mutant mice carrying conditional knockout alleles of Bap1, Nf2, p19Arf and knock-out alleles of Cdkn2a, Cdkn2b have been generated that provide an excellent mouse model for mesothelioma. Introducing these lesions in the mesothelial lining of the thoracic cavity results in rapid mesothelioma development with a marker profile and drug response closely mimicking human mesothelioma, making this model particular suitable for studying the biology of mesothelioma and for serving as an intervention model to test new treatment protocols.

Recently, a large drug screen resulted in the identification of multiple single drugs, including TRAIL inhibitor, 5-FU, Plk1/2/3 inhibitor, MDM2 inhibitor, Bcl-2, Bcl-xl inhibitor, and GSK3B inhibitor, that were found potent in killing mesothelioma cells with BAP1 mutations [Kolluri et al., 2018, eLIFE 7: e30224]. Additionally, EZH2 inhibition has been described as being effective in mesothelioma cells with BAP1 loss [LaFave et al., 2015, Nat Med 21: 1344-1349], while a subgroup of mesothelioma cells with BAP1 loss may be sensitive to FGFR inhibition [Quispel-Janssen et al., 2018. Clin Cancer Res 24: 84-94]. Yet other reports indicate that loss of BAP1 renders cells hypersensitive to HDAC inhibitors [Sacco et al., 2015, Oncotarget 6: 13757-13771], or to PARP inhibition [Parrotta et al., 2017, J Thorac Oncol 12: 1309-1319]. No rationale has been proposed to combine any of these inhibitors.

Finally, a combination of gemcitabine and cisplatin has been reported to be effective in killing mesothelioma cells, especially mespothelioma cells with loss of BAP1 [Gauzzelli et al., 2019. Int J Mol Sci 19: 20-22, Hassan et al., 2019. PNAS 30: 9008-9013]. The invention provides a combination of a fibroblast growth factor receptor (FGFR) inhibitor and an Enhancer of zeste homolog 2 (EZH2) inhibitor for use in a method of treating a patient suffering from a BReast CAncer susceptibility gene 1 (BRCAD-associated protein 1 (BAP1) negative cancer, especially BAP1-negative mesothelioma. It was found in the present study that EZH2 inhibition in mesothelioma cells results in upregulation of key molecules in the FGF pathway, including FGF2, FGF7, and FGFR2. Therefore, FGFR was thought to be a suitable target to combine with EZH2 inhibition. Other combinations such as EZH2i and PARPi, EZH2i and Bcl2i, were found not to be effective.

Surprisingly, a combination of FGFR and EZH2 inhibitors was found to be active against mesothelioma cells, especially mesothelioma cells that are BAP1-mutant, at much lower concentrations than the individual drugs tested alone, suggesting that a combination of EZH2 inhibitor and FGFR inhibitor is highly synergistic.

Said FGFR inhibitor preferably is a selective FGFR1, FGFR2, and/or FGFR3 inhibitor. A preferred FGFR inhibitor is AZD4547.

Said EZH2 inhibitor preferably is selected from EPZ-6438, GSK126 and/or CPI-1205, preferably GSK126.

In an embodiment, said patient has previously been treated with one or more chemotherapeutical agents, preferably with cisplatin or carboplatin, in combination with either pemetrexed or raltitrexed, and may have become resistant to said one or more chemotherapeutical agents.

The combination of a FGFR inhibitor and an EZH2 inhibitor for use according to the invention preferably is provided to the patient in cycles of 2-6 weeks, with at least one day of rest in between the cycles. The number of cycles preferably is 4-10.

The combination of a FGFR inhibitor and an EZH2 inhibitor for use according to the invention may be combined or alternated with a G2 checkpoint abrogator, a focal adhesion kinase inhibitor and/or gemcitabine.

The invention further provides a pharmaceutical preparation, comprising a FGFR inhibitor and an EZH2 inhibitor. Said pharmaceutical preparation may comprises a pharmaceutical preparation comprising a FGFR inhibitor and a pharmaceutical preparation comprising an EZH2 inhibitor. Said pharmaceutical preparation is for use in a method of treating BRCA1-associated protein 1 (BAP1) negative cancer, especially BAP1-negative mesothelioma.

The invention further provides a method of identifying a patient with cancer, especially mesothelioma, eligible for treatment with a combination of a FGFR inhibitor and an EZH2 inhibitor, comprising testing a biological sample comprising cancer cells from the patient for the presence of a mutation in BAP1, wherein the patient is eligible for treatment with said combination if said tumor cells in said sample test positive for a mutation in BAP1, and/or loss of BAP1.

A preferred method of identifying a patient with cancer, especially mesothelioma, eligible for treatment with a combination of a FGFR inhibitor and an EZH2 inhibitor comprises (a) providing a bodily fluid from the patient, (b) magnetically separating cancer cells from essentially all other cells in said sample using magnetic nanoparticles comprising antibodies that target cancer cells, and (c) determining the presence of a mutation in BAP1, and/or loss of BAP1, in cancer cells in said cancer cell-enriched sample, preferably by immunohistochemistry.

The invention further provides a mouse model for mesothelioma, comprising knock-out alleles of Cdkn2a and Cdkn2b, and conditional knockout alleles of Bap1, Nf2 and p19Arf that can be activated at least in the mesothelial lining of the thoracic cavity.

### FIGURE LEGENDS

FIG. 1: BAP1 accelerates tumor initiation and progression in a mouse model of mesothelioma. A. CBioportal oncoplot of mesothelioma TCGA dataset of frequent genetic alterations such as BAP1, NF2, CDKN2A, CDKN2B. B. Overall survival of Ad-CMV-Cre activated Bap 1f/f mice (n=13), Bap 1f/f;Nf2f/f;Cdkn2ab (n=13) and Nf2;Cdkn2ab (n=24). C: H&E staining showing epithelioid and spindle cells and immunohistochemistry showing markers of mesothelioma such as cytokeratins, WT1, and podoplanin.

FIG. 2: Mesothelioma show hyperactivation of MAPK and PI3K pathways and inflammatory tumor microenvironment. A. Representative IHC staining of P-ERK, P-AKT, P-S6K and YAP/TAZ of mouse mesothelioma. B. Representative IHC staining of F-4/80, FOXP3, CD3 and EZH2 of mouse mesothelioma.

FIG. 3: BAP deficient mesothelioma cells show a repressive transcriptional program. A. Genomic distribution of H3K27me3 binding in Bap1 deficient and proficient mouse mesothelioma tumor cells. B. MA plot showing differential expression of Bap1 deficient and proficient mouse mesothelioma tumor cells showing profound downregulation in Bap1 deficient mesothelioma.

FIG. 4: Mouse mesothelioma cells show similar drug response profile to human mesothelioma. Dose response curve of BAP1 deficient and proficient mesothelioma cells to A. IR-radiation. B. FGFR inhibition. C. EZH2 inhibition. D. Clonogenicity assay of Bap1 deficient and proficient mesothelioma cells upon EZH2 inhibition by shRNA. E. Upregulation of FGF7, FGF2 and FGFR2 upon treatment of Bap1 deficient cells with EZH2 inhibitor GSK126 as presented by log 2 expression of DMSO or GSK126 treated mouse mesothelioma cells.

FIG. 5: FGFR inhibition and EZH2 inhibition is a synergistic combination. A. Colony formation assay shows combination of AZD4547 and GSK126 completely kill Bap1 deficient mouse mesothelioma cells, whereas Bap1 proficient cells are less sensitive to the combination. B. 3-dimensional MaeSynergy™ plot showing synergy score (a.u.) across the concentration range used for the combination of AZD4547 and GSK126. C. Tumor burden in thoracic wall in vehicle and GSK126 and AZD4547 treated mesothelioma mouse model (BNC mouse). Y axis represents tumor burden score, whereby a score of 10 represents highest tumor burden in that cohort, while a score of 0 indicates no tumor.

FIG. 6: Mesothelioma mouse shows improved survival upon cisplatin and pemetrexed treatment. A. Survival curve of vehicle and cisplatin-pemetrexed treated mouse. B. Caspase-3 and Ki-67 staining of treated and untreated mice tumors.

FIG. 7: 3-dimensional MaeSynergy™ plot with synergy score representing percent inhibition over the predicted effect over a range of concentrations in matrix in BNC mouse mesothelioma cells. A. Synergy score of 279 indicates very weak synergy. B. Synergy score of 120 indicates no synergy.

### 4 DETAILED DESCRIPTION OF THE INVENTION

### 4.1 Definitions

The term “effective amount”, as used herein, means an amount of a pharmaceutical compound, such as a FGFR inhibitor and a EZH2 inhibitor, that produces an effect on the cancer to be treated.

The term “BAP1 negative mesothelioma” as used herein, refers to a mesothelioma having a reduced activity, including absence of activity, of the product of tumor suppressor gene BRCA-associated protein 1 (BAP1).

The term “FGFR inhibitor” as used herein, refers to a molecule that inhibits signal transduction from a Fibroblast Growth Factor receptor. Said molecule preferably inhibits kinase activity of said FGFR. A preferred molecule is selective for FGFR, when compared to other tyrosine kinases, meaning that the molecule is at least two times more potent in inhibiting a FGFR, when compared to other tyrosine kinases. A preferred FGFR inhibitor inhibits FGFR1, FGFR2, and/or FGFR3.

The term “EZH2 inhibitor” as used herein, refers to a molecule that inhibits a histone-lysine N-methyltransferase enzyme (EC 2.1.1.43) of Polycomb repressive complex 2. EZH2 methylates lysine 27 of histone H3 to promote transcriptional silencing. A preferred molecule is selective for EZH2, when compared to other histone methylases, including other members of the SET domain family of lysine methyltransferases, meaning that the molecule is at least two times more potent in inhibiting EZH2, when compared to other histone methylases including EZH1. The term “combination”, as is used herein, refers to the administration of effective amounts of a FGFR inhibitor and an EZH2 inhibitor to a patient in need thereof. Said FGFR inhibitor and EZH2 inhibitor may be provided in one pharmaceutical preparation, or as two distinct pharmaceutical preparations. When administered as two distinct pharmaceutical preparations, they may be administered on the same day or on different days to a patient in need thereof, and using a similar or dissimilar administration protocol, e.g. daily, twice daily, biweekly, orally and/or by infusion. Said combination is preferably administered repeatedly according to a protocol that depends on the patient to be treated (age, weight, treatment history, etc.), which can be determined by a skilled physician.

The term “PCR”, as is used herein, refers to an amplification reaction that is characterized by repeated cycles of denaturation of target nucleic acid template, annealing of primers, and extension (synthesis) of new nucleic acid strand. The specificity of a PCR reaction is substantially determined by the % identity of the primers to the target nucleic acid template and the annealing temperature. The term “real-time PCR reaction”, as is used herein, refers to a PCR amplification reaction to which a labeled probe or a dye is added to generate a signal. The intensity of the signal is a measure for the amount of product that is generated. Detection of the signal in real-time allows quantification of the amount of starting material. A real-time PCR reaction is performed in specialized thermal cyders with detection systems that detect the signal, for example a LightCycler 48011 (Roche Diagnostics, Almere, The Netherlands), a Mastercycler Realplex Ep Real-Time PCR System (Eppendorf A.G., Hamburg, Germany), or a StepOne™ Plus (Thermo Fisher Scientific Inc., Waltham, Mass. USA). However, a separate probe does not need to be present. Some real-time PCR reactions incorporate a dye in the primer (e.g. Scorpion® primers; Premier Biosoft, Palo Alto, Calif., USA) and are included herein.

The terms “forward primer” and “reverse primer”, as are used herein, refer to a single-stranded oligonucleotide or oligonucleotide mimic of 15-50 bases, preferably 16-30 bases, that is complementary to nucleic acid sequences flanking the region to be amplified. The sequence of the forward primer and reverse primer determines the specificity of the amplification reaction. Preferred primers are preferably about 100% identical to a region on a target nucleic acid template such that only the region between two primers in a target nucleic acid template is amplified. The distance between the primer binding sites on the target nucleic acid template will determine the size of the amplified product.

The term “probe”, as is used herein, refers to a single-stranded oligonucleotide or oligonucleotide mimic of 15-50 bases, preferably 16-30 bases, that is complementary to a nucleic acid sequence within a target nucleic acid, such as a PCR amplicon. A preferred probe is about 100% identical to the target region of a target nucleic acid. A probe generally comprises a detectable label at its 3′- or 5′-end.

The terms “target DNA molecule”, “target nucleic acid (template)” and “template DNA molecule”, as are used herein, refer to nucleic acid of which a region between two primers, preferably a forward primer and a reverse primer, is amplified. A target nucleic acid template is a gene or a gene product, such as a RNA product, or a part of the gene or part of the gene product.

The term “amplicon”, as is used herein, refers to a region on a target nucleic acid template that is amplified using said two primers, preferably a forward primer and a reverse primer. An amplicon preferably comprises a nucleic acid sequence that is complementary to a nucleic acid sequence of a probe that specifically recognizes said amplicon.

The term “specifically hybridize”, as is used herein, refers to nucleic acid molecules that form a double stranded nucleic acid molecule under stringent conditions.

The terms “stringency” and “stringent hybridization” refer to hybridization conditions that affect the stability of hybrids, e.g., temperature, salt concentration, pH, and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g. at least 2-fold over background). Stringent conditions may be sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridizes to a perfectly matched probe or primer. Hybridization procedures are well known in the art and are described by e.g. Ausubel et al., 1998. Current Protocols in Molecular Biology, John Wiley, New York; and Sambrook et al., 2001. Molecular cloning: a laboratory manual. Cold Spring Harbor Laboratory Press, New York.

The term “blood”, as is used herein, includes reference to serum and plasma. The terms serum and plasma both refer to blood components without cells, whereby serum also excludes clotting factors such as fibrinogen. As is known to a person skilled in the art blood may, for example, be centrifuged to remove cellular components. The thus obtained plasma may be coagulated followed by, for example, centrifugation to remove the clotting factors, resulting in serum.

### 4.2 Methods of Treatment

The invention relates to the use of one or more biomarkers to evaluate the therapeutic effectiveness of treating a cancer patient with an EZH2 inhibitor, in combination with a FGFR inhibitor. It is based, at least in part, on the discovery that loss of BAP1 results in upregulation of EZH2 expression and increased activity of the MAPK pathways. Surprisingly, it was found that inhibition of EZH2 results in over-activity of FGF receptors due to upregulation of key FGF pathway components such as FGF2, FGF7 and FGFR2. Therefore, targeting both pathways would unleash greater therapeutic activity.

The cancer is a BAP1-negative cancer, such as a BAP1-negative uveal melanoma, a BAP1-negative renal cell carcinoma, a BAP1-negative lung cancer, a BAP1-negative breast cancer, a BAP1-negative cholangiocarcinoma, a BAP1-negative pancreatic cancer, melanocytic BAP 1 mutated atypical intradermal tumors, and a BAP1-negative mesothelioma.

A method for determining presence of BAP1-negative cancer cells, comprises providing a biological sample from a cancer patient, said sample comprising cancer cells, and determining the presence of a mutation in BAP1 and/or loss of BAP1, in said cancer cells, preferably by immunohistochemistry.

The protein product Bap 1 is normally localized to the nucleus. Whether a cancer cell is BAP1 negative, is preferably determined by immunohistochemistry, in which Bap1-negative cells are scored as having a reduced or undetectable nuclear immunoreactivity using appropriate anti-Bap 1 antibodies. Further methods comprise detection of a truncated BAP1 mRNA and/or truncated Bap1 protein, and/or reduced expression of BAP1 mRNA and/or Bap1 protein.

A preferred method for determining whether a cancer cell is BAP1 negative, involves amplification of nucleic acid material, preferably polymerase chain reaction (PCR). For example, a reduced expression of BAP1 can be determined by a difference in the Cq value (i.e. the quantitation cycle or the cycle in which a detectable (fluorescence) signal is obtained) between BAP1 and a reference transcript or gene, such as the Cq value obtained for the markers cytokeratin 5/6, calretinin, WT-1 and/or podoplanin. As an alternative, or in addition, specific mutations within the BAP1 gene can be detected by PCR using specific primers as forward and reverse primer that span the mutated or deleted region, and an appropriate probe, as is known by a person skilled in the art.

Said biological sample comprising cancer cells preferably is obtained from a cancerous growth, or of a tumor suspected to be cancerous, depending on the size of the cancerous growth. A cancerous growth can be removed by surgery including, for example, lumpectomy, lap aroscopic surgery, colostomy, lobectomy, bilobectomy or pneumonectomy. Said biological sample can also be derived by biopsy, comprising aspiration biopsy, needle biopsy, incisional biopsy, and excisional biopsy. It is preferred that at least 10% of the cells in the biological sample are cancer cells, more preferred at least 20%, and most preferred at least 30%. Said percentage of cancer cells can be determined by analysis of a stained section, for example a hematoxylin and eosin-stained section, from the cancerous growth. Said analysis can be performed or confirmed by a pathologist.

As an alternative, said biological sample comprising cancer cells is obtained from a bodily fluid from the cancer patient. After provision of a bodily fluid from the patient, cancer cells may be enriched, for example, by magnetically separating cancer cells from essentially all other cells in said sample using magnetic nanoparticles comprising antibodies that target cancer cells, followed by determining presence or absence of BAP1-negative cancer cells in said cancer cell-enriched sample.

The BAP1 gene is localized at 3p21.3, a region of loss of heterozygosity for breast cancer as well as a region frequently deleted in lung carcinomas (Jensen et al., 1998. Oncogene 16: 1097-1112). BAP1 was found to be somatically mutated in 24 (14%) of 176 tumors, and most mutations were predicted to truncate the protein (Pena-Llopis et al., 2012. Nature Genet 44: 751-759). Known BAP1 mutations include intragenic homozygous rearrangements and deletions of BAP1, including loss of heterozygosity for the BAP1 gene, frameshift mutations including a 1-bp deletion (1305delG) in exon 13 of the BAP1 gene and a heterozygous A-to-G transition removing the acceptor splice site of the last exon (2057-2A-G) (Wiesner et al., 2011. Nature Genet 43: 1018-1021); an A-to-G transition in the splice acceptor site of intron 6 of the BAP1 gene, a C-to-T transition in exon 16 of the BAP1 gene, resulting in a gln684-to-ter (Q684X) substitution, and a 1-bp deletion (1832delC) in exon 13 of the BAP1 gene, resulting in a frameshift and truncation upstream of the Bap1 nuclear localization signal (Testa et al., 2011. Nature Genet 43: 1022-1025); a 1-bp deletion (c.1654delG) in the BAP1 gene, resulting in a frameshift and premature termination (Asp552IlefsTer19), and a 2-bp deletion (c.78_79 delGG) in the BAP1 gene (Popova et al., 2013. Am J Hum Genet 92: 974-980); and a 799C-T transition in the BAP1 gene, resulting in a gln267-to-ter (Q267X) (Abdel-Rahman et al., 2011. J Med Genet 48: 856-859). An overview of mutations in BAP1 is provided in Liu et al., 2018 (Liu et al., 2018. Mol Genet Genomic Med 1-14; 10.1002/mgg3.458).

The invention provides a combination of a FGFR inhibitor and an EZH2 inhibitor for use in a method of treating a patient suffering from a BRCA1-associated protein 1 (BAP1) negative cancer, such as a BAP1-negative uveal melanoma, a BAP1-negative renal cell carcinoma, a BAP1-negative lung cancer, a BAP1-negative breast cancer, a BAP1-negative cholangiocarcinoma, a BAP1-negative pancreatic cancer, melanocytic BAP 1 mutated atypical intradermal tumors, and a BAP1-negative mesothelioma.

The invention further provides a FGFR inhibitor for use in a method of treatment of a BAP1-negative cancer, whereby said treatment further comprises an EZH2 inhibitor.

The invention further provides an EZH2 inhibitor for use in a method of treatment of a BAP1-negative cancer, whereby said treatment further comprises a FGFR inhibitor

Known FGFR inhibitors that may be combined with an EZH2 inhibitor for use according to the invention include Ponatinib (AP24534; ARIAD Pharmaceuticals, Inc.; 3-(2-imidazo[1,2-b]pyridazin-3-ylethynyl)-4-methyl-N-[4-[(4-methylpiperazin-1-yl)methyl]-3-(trifluoromethyl)phenyl]benzamide), which is provided at a daily dosage of 10-100 mg, preferably at about 45 mg once daily; Nintedanib (BIBF 1120; Boehringer Ingelheim Pharmaceuticals; methyl (3Z)-3-[[4-[methyl-[2-(4-methylpiperazin-1-yl)acetyl]amino]anilino]-phenylmethylidene]-2-oxo-1H-indole-6-carboxylate), which is provided at a daily dosage of 20-250 mg, preferably at 100-150 mg; Dovitinib (Novartis Pharmaceuticals; (3Z)-4-amino-5-fluoro-3-[5-(4-methylpiperazin-1-yl)-1,3-dihydrobenzimidazol-2-ylidene]quinolin-2-one), which is provided at a daily dosage of 100-750 mg, preferably at 200 to 500 mg daily: Brivanib (BMS-540215; Bristol-Myers Squibb; [(2R)-1-[4-[(4-fluoro-2-methyl-1H-indol-5-yl)oxy]-5-methylpyrrolo[2,1-f][1,2,4]triazin-6-yl]oxypropan-2-yl](2S)-2-aminopropanoate), which is provided at a daily dosage of 200-2000 mg, preferably at about 800 mg daily; Derazantinib (ARQ-087; ArQule, Inc.; (GR)-6-(2-fluorophenyl)-N-[3-[2-(2-methoxyethylamino)ethyl]phenyl]-5,6-dihydrobenzo[h]quinazolin-2-amine), which is provided at a daily dosage of 100-500 mg, preferably at about 300 mg daily: PRN1371 (BLU9931; Principia Biopharma; 6-(2,6-dichloro-3,5-dimethoxyphenyl)-2-(methylamino)-8-[3-(4-prop-2-enoylpiperazin-1-yl)propyl]pyrido[2,3-d]pyrimidin-7-one); S49076 (Servier; 3-[[(3Z)-3-[[4-(morpholin-4-ylmethyl)-1H-pyrrol-2-yl]methylidene]-2-oxo-1H-indol-5-yl]methyl]-1,3-thiazolidine-2,4-dione), which is provided at a daily dosage of 15-900 mg, preferably at 200-300 mg daily; CH5183284 (Debio-1347; Debiopharm International SA: [5-amino-1-(2-methyl-3H-benzimidazol-5-yl)pyrazol-4-yl]-(1H-indol-2-yl)methanone), which is provided at a daily dosage of 2-50 mg, preferably at 10-30 mg daily; LY2874455 (Eli Lilly; 2-[4-[(E)-2-[5-[(1R)-1-(3,5-dichloropyridin-4-yl)ethoxy]-1H-indazol-3-yl]ethenyl]pyrazol-1-yl]ethanol), which is provided at a daily dosage of 1-1000 mg, preferably at 2-200 mg daily; TAS-120 (Taiho Pharmaceutical; 1-[(3S)-3-[4-amino-3-[2-(3,5-dimethoxyphenyl)ethynyl]pyrazolo[3,4-d]pyrimidin-1-yl]pyrrolidin-1-yl]prop-2-en-1-one), which is provided at a daily dosage of 2-500 mg, preferably at 8-200 mg daily; and BAY-1163877 (Rogaratinib; Bayer AG; 1-tert-butyl-3-[2-[4-(diethylamino)butylamino]-6-(3,5-dimethoxyphenyl)pyrido[2,3-d]pyrimidin-7-yl]urea), which is provided at a daily dosage of 10-500 mg, preferably at about 200 mg daily.

Further known FGF pathway inhibitors include antibodies such as MFGR1877S (Fauvel and Yasri, 2014. MAbs 6: 838-851) and a fusion protein between FGFR1 and the Fc portion of a human IgG1 (FP-1039; Five Prime Therapeutics: also known as GSK230), that acts as a ligand trap by sequestering fibroblast growth factors involved in tumor growth (Harding et al., 2013. Science Transl Med 5: pp. 178ra39).

Said FGFR inhibitor preferably is selective for FGFR1, FGFR2, and/or FGFR3. Said selective FGFR inhibitor preferably is selected from BGJ398 (Infigratinib; Novartis; 3-(2,6-dichloro-3,5-dimethoxyphenyl)-1-[6-[4-(4-ethylpiperazin-1-yl)anilino]pyrimidin-4-yl]-1-methylurea), which is provided at a daily dosage of 10-250 mg, preferably at 50-100 mg daily; JNJ 42756493 (Erdafitinib; Janssen; N′-(3,5-dimethoxyphenyl)-N′-[3-(1-methylpyrazol-4-yl)quinoxalin-6-yl]-N-propan-2-ylethane-1,2-diamine), which is provided at a daily dosage of 2-10 mg, preferably at 8-9 mg daily; AZD4547 (AstraZeneca; N-[5-[2-(3,5-dimethoxyphenyl)ethyl]-1H-pyrazol-3-yl]-4-[(3R,5S)-3,5-dimethylpiperazin-1-yl]benzamide), which is provided at a daily dosage of 50-500 mg, preferably at 100-200 mg daily, and/or PD173074 (N-[2-[[4-(Diethylamino)butyl]amino-6-(3,5-dimethoxyphenyl)pyrido[2,3-d]pyrimidin-7-yl]-N′-(1,1-dimethylethyl)urea), which is provided at a daily dosage of 50-100 mg.

Known EZH2 inhibitors that may be combined with a FGFR inhibitor for use according to the invention include DZNep ((1S,2R,5R)-5-(4-aminoimidazo[4,5-c]pyridin-1-yl)-3-(hydroxymethyl)cyclopent-3-ene-1,2-diol): Eli (6-cyano-N-[(4,6-dimethyl-2-oxo-1H-pyridin-3-yl)methyl]-1-pentan-3-ylindole-4-carboxamide); EPZ005687 (Epizyme, Inc.; 1-cyclopentyl-N-[(4,6-dimethyl-2-oxo-1H-pyridin-3-yl)methyl]-6-[4-(morpholin-4-ylmethyl)phenyl]indazole-4-carboxamide); UNC1999 (N-[(6-methyl-2-oxo-4-propyl-1H-pyridin-3-yl)methyl]-1-propan-2-yl-6-[6-(4-propan-2-ylpiperazin-1-yl)pyridin-3-yl]indazole-4-carboxamide); a dual EZH1 and EZH2 inhibitor such as Daiichi-Sankyo's DS-3201 and/or DS32013201b; sinefungin (2S,5S)-2,5-diamino-6-[(2R,3S,4R,5R)-5-(6-aminopurin-9-yl)-3,4-dihydroxyoxolan-2-yl]hexanoic acid; Dong et al., 2015. Chem Res Toxicol; 28: 2419-25); and GSK343 (GlaxoSmithKline; N-[(6-methyl-2-oxo-4-propyl-1H-pyridin-3-yl)methyl]-6-[2-(4-methylpiperazin-1-yl)pyridin-4-yl]-1-propan-2-ylindazole-4-carboxamide).

Preferred EZH2 inhibitors that may be combined with a FGFR inhibitor for use according to the invention are provided by GSK126 (GlaxoSmithKline; 1-[(2S)-butan-2-yl]-N-[(4,6-dimethyl-2-oxo-1H-pyridin-3-yl)methyl]-3-methyl-6-(6-piperazin-1-ylpyridin-3-yl)indole-4-carboxamide), which is provided by biweekly infusion of 10-3000 mg, preferably at 50-1000 mg; EPZ-6438 (Tazemetostat; Epizyme, Inc.; N-[(4,6-dimethyl-2-oxo-1H-pyridin-3-yl)methyl]-3-[ethyl(oxan-4-yl)amino]-2-methyl-5-[4-(morpholin-4-ylmethyl)phenyl]benzamide), which is provided at a daily dosage of 500-3000 mg, preferably at 1500-2000 mg daily; and CPI-1205 (Constellation Pharmaceuticals; N-[(4-methoxy-6-methyl-2-oxo-1H-pyridin-3-yl)methyl]-2-methyl-1-[(1R)-1-[1-(2,2,2-trifluoroethyl)piperidin-4-yl]ethyl]indole-3-carboxamide), which is provided at a daily dosage of 100-300 mg/kg, preferably at about 200 mg/kg.

Said preferred EZH2 inhibitor selected from GSK126, EPZ-6438 and CPI-1205 preferably is combined with a FGFR inhibitor, preferably a selective FGFR1, FGFR2, and/or FGFR3 inhibitor. Preferred combinations include BGJ398 and GSK126, JNJ 42756493 and GSK126, AZD4547 and GSK126, BGJ398 and EPZ-6438, JNJ 42756493 and EPZ-6438, AZD4547 and EPZ-6438, BGJ398 and CPI-1205, JNJ 42756493 and CPI-1205, and AZD4547 and CPI-1205.

Said combination may be provided in one pharmaceutical preparation, or as two distinct pharmaceutical preparations. Said single or distinct pharmaceutical preparations further comprise pharmaceutically acceptable excipients, as is known to a person skilled in the art. A preferred pharmaceutical preparation is provided by a tablet. The term “tablet” encompasses a “capsule” and a “caplet”.

Pharmaceutically acceptable excipients include diluents, binders or granulating ingredients, a carbohydrate such as starch, a starch derivative such as starch acetate and/or maltodextrin, a polyol such as xylitol, sorbitol and/or mannitol, lactose such as α-lactose monohydrate, anhydrous α-lactose, anhydrous β-lactose, spray-dried lactose, and/or agglomerated lactose, a sugar such as dextrose, maltose, dextrate and/or inulin, or combinations thereof, glidants (flow aids) and lubricants to ensure efficient tableting, and sweeteners or flavours to enhance taste.

Said pharmaceutical preparation according to the invention is for use in a method of treating BRCA1-associated protein 1 (BAP1) negative cancer, especially BAP1-negative mesothelioma.

A combination of an EZH2 inhibitor and a FGFR inhibitor, preferably a selective FGFR1, FGFR2, and/or FGFR3 inhibitor, preferably is for use in a method of treatment of a cancer patient who has previously been treated with one or more of chemotherapeutical agents, including cytotoxic agents, immunomodulating agents and immunotoxic agents. Said one or more chemotherapeutical agents include alkylating agents such as busulfan, melphalan, carboplatin, cisplatin, cyclophosphamide, dacarbazine, carmustine, nimustin, lomustine, ifosfamide, temozolomide, navelbine and altretamine, antibiotics such as leomycin, doxorubicin, adriamycin, idarubicin, epirubicin and plicamycin, antimetabolites such as sulfonamides, folic acid antagonists, gemcitabine, 5-fluorouracil (5-FU), leucovorine, leucovorine with 5-FU, 5-FU with calcium folinate and leucovorin, capecitabine, mercaptopurine, cladribine, pentostatine, methotrexate, raltitrexed, pemetrexed, thioguanine, and camptothecin derivates such as topotecan and irinotecan, hormones and antagonists thereof such as flutamide, goserelin, mitotane and tamoxifen, mustard gas derivatives such as melphalan, carmustine and nitrogen mustard, and alkaloids such as taxanes, docetaxel, paclitaxel, etoposide, vincristine, vinblastine and vinorelbine.

Said combination of an EZH2 inhibitor and a FGFR inhibitor preferably is for use in a method of treatment of a cancer patient who has previously been treated with cisplatin or carboplatin, in combination with either pemetrexed or raltitrexed.

Said combination of an EZH2 inhibitor and a FGFR inhibitor preferably provides a second line therapy for a cancer patient that has become resistant to first line therapy comprising said one or more chemotherapeutical agents, especially who has become resistant to first line therapy comprising cisplatin or carboplatin, in combination with either pemetrexed or raltitrexed.

Said combination of an EZH2 inhibitor and a FGFR inhibitor for use according to the invention may be provided to a patient in cycles of 1-6 weeks, with at least one day of rest in between the cycles. Said cycles preferably are spaced apart by at least two days, such as three days, four days, one week or two weeks. The total number of cycles preferably is 4-10.

Said combination of an EZH2 inhibitor and a FGFR inhibitor for use according to the invention may be combined or alternated with a G2 checkpoint abrogator such as SB-218078 (9,10,11,12-tetrahydro-9,12-epoxy-1H-diindolo[1,2,3-fg:3′,2′,1′-kl]pyrrolo[3,4-i][1,6]benzodiazocine-1,3(2H)-dione) and UCN-01 ((3R,9S,10R,11R,13R)-2,3,10,11,12,13-hexahydro-3-hydroxy-10-methoxy-9-methyl-11-(methylamino)-9,13-epoxy-1H,9H-diindolo[1,2,3-gh:3′,2′,1′-lm]pyrrolo[3,4-j][1,7]benzodiazonin-1-one); a focal adhesion kinase inhibitor such as amlodipine besylate (benzenesulfonic acid;3-O-ethyl 5-O-methyl 2-(2-aminoethoxymethyl)-4-(2-chlorophenyl)-6-methyl-1,4-dihydropyridine-3,5-dicarboxylate), defactinib (N-methyl-4-[[4-[[3-[methyl(methylsulfonyl)amino]pyrazin-2-yl]methylamino]-5-(trifluoromethyl)pyrimidin-2-yl]amino]benzamide), or GSK2256098 (2-[[5-chloro-2-[(5-methyl-2-propan-2-ylpyrazol-3-yl)amino]pyridin-4-yl]amino]-N-methoxybenzamide); and/or an antimetabolite such as sulfonamides, folic acid antagonists, gemcitabine, 5-fluorouracil (5-FU), leucovorine, leucovorine with 5-FU, 5-FU with capecitabine, mercaptopurine, cladribine, pentostatine, methotrexate, raltitrexed, pemetrexed, thioguanine, and camptothecin derivates such as topotecan and irinotecan.

The invention further provides a method of treating a patient suffering from a BRCA1-associated protein 1 (BAP1) negative cancer, especially a BAP1-negative mesothelioma, comprising providing an effective amount of a FGFR inhibitor and an effective amount of an EZH2 inhibitor to said patient. Said method may include the features indicated herein above.

The invention further provides an use of a FGFR inhibitor in the preparation of a medicament for treatment of a patient suffering from a BRCA1-associated protein 1 (BAP1) negative cancer, especially BAP1-negative mesothelioma, wherein said treatment is combined with an EZH2 inhibitor. Said use may include the features indicated herein above.

The invention further provides an use of an EZH2 inhibitor in the preparation of a medicament for treatment of a patient suffering from a BRCA1-associated protein 1 (BAP1) negative cancer, especially BAP1-negative mesothelioma, wherein said treatment is combined with a FGFR inhibitor. Said use may include the features indicated herein above.

The invention further provides a method of identifying a patient with cancer, especially mesothelioma, eligible for treatment with a combination of a FGFR inhibitor and an EZH2 inhibitor, comprising testing a biological sample comprising cancer cells from the patient for the presence of a mutation in BAP1, wherein the patient is eligible for treatment with said combination if said tumor cells in said sample test positive for a mutation in BAP1.

As is indicated herein above, said biological sample comprising cancer cells preferably is obtained from a cancerous growth, or of a tumor suspected to be cancerous, depending on the size of the cancerous growth.

As an alternative, said biological sample comprising cancer cells may be provided as a biological fluid. In a preferred embodiment, a cancer cell-enriched sample is obtained from said biological fluid, for example by magnetically separating cancer cells from essentially all other cells in said sample using magnetic nanoparticles comprising binding molecules such as antibodies that target said cancer cells in the biological fluid. Systems for enrichment of circulating cancer cells are known in the art and include CellSearcht (Veridex, Raritan, NJ), CellCollector@ (GILUPI GmbH, Potsdam, Germany), and Surface-Enhanced Raman Scattering (SERS)-based systems, including SERS-active nanoparticles (Garza and Cote, 2017. Anal Chem 89: 13120-13127). Markers that are currently used for enrichment of cancer cells from a biological fluid include epithelial cell adhesion molecule (EpCAM), which is present on cancer cells from lung, breast, bladder cancer and cholangiocarcinoma (Man et al., 2011. J Clinic Experiment Pathol 1: 102; Yang et al., 2016. Hepatology 63: 148-158), EphB4, Epidermal Growth Factor Receptor, and/or human epidermal growth factor receptor 2 (HER2), which are present on cancer cells from breast cancer (Man et al., 2011. J Clinic Experiment Pathol 1: 102), melanoma antigen recognized by T cells-1 (MART1), gp100, CD146, and/or Melanoma Antigens Gene-3 (MAGE-3), which are present on cancer cells from melanoma cancer (Rapanotti et al., 2017. Cell Death Discov 3: 17005), CD45 and/or keratin 8, which are present on cancer cells from renal cell carcinoma (Maertens et al., 2017. Oncotarget 8: 87710-87717), EpCAM, combined with size-dependent filtration, for enrichment of cancer cells from pancreatic cancer (Brychta et al., 2017. Oncotarget 8): 86143-86156), MART1, which is present on melanocytic cancer cells (Prieto and Shea, 2011. Arch Pathol Lab Med 135: 853-859), and mesothelin and osteopontin, which are present on cancer cells from mesothelioma (Bruno et al., 2018. Euro J Surg Oncol 44: 792-798).

Immunohistochemistry can be used to detect the expression and/or presence of a biomarker, e.g., in a biopsy sample. A suitable antibody can be brought into contact with, for example, a thin layer of cells, followed by washing to remove unbound antibody, and then contacted with a second, labeled, antibody. Labeling can be by fluorescent markers, enzymes, such as peroxidase, avidin or radiolabeling. The assay can be scored visually, using microscopy, and the results can be quantitated.

### 4.3 Mouse Models for Human Mesothelioma

Mesothelioma is a highly aggressive tumor of serosal surfaces. The majority of cases are linked to asbestos exposure and it takes as long as 30-50 years for tumors to arise from the time of exposure. Inflammation is a hallmark of asbestos deposition in tissue and contributes to carcinogenesis. Mesothelioma is an aggressive disease with poor prognosis and limited treatment options. The only first line therapy for a patient with unresectable disease is cisplatin or carboplatin, in combination with pemetrexed or raltitrexed, with limited efficacy. Currently, there are no approved targeted therapies for mesothelioma. Therefore, there is an urgent need of novel therapy for this deadly disease.

Human mesothelioma shows frequent inactivation of the CDKN2AB locus that encodes for p16INK4A, p15INK4B, and p14ARF, three cell cycle inhibitors and Neurofibromatosis Type 2 (NF2) with well-established tumor suppressor function. Recently, the BRCA1 associated protein 1 (BAP1) gene has been found mutated, deleted or epigenetically silenced in human mesothelioma.

The polycomb group (PcG) proteins are highly conserved complexes, which function as chromatin modifiers and transcriptional regulators. PcG proteins carry out their function via three major complexes: (1) polycomb repressive complex 1 (PRC1) involved mainly in gene repression via mono-ubiquitination of lysine 119 of H2A (H2A119); (2) polycomb repressive deubiquitinase (PR-DUB) opposes the function of PRC1 via deubiquitination of H2A119 and PRC2 which modifies chromatin via depositing a repressive histone mark; AND (3) Histone H3 lysine 27 tri-methylation (H3K27me3).

Bap1 removes ubiquitin from histone H2A119 and is linked to the PRC complex, PR-DUB. BAP1 is altered in 60-70% cases of mesothelioma and its germline mutation has been shown to predispose for mesothelioma. However, how precisely BAP1 loss contributes to aggressive mesothelioma is not yet known. Recently, its role in myeloid transformation was reported. In myeloid cells, BAP1 loss has been shown to result in elevated levels of EZH2 and H3K27me3 along with responsiveness to pharmacological inhibition of EZH2. Recent literature suggests that Bap1 promotes DNA double-strand break (DSB) repair and mutations in BAP1 impair this function. Being a member of the PR-DUB complex, it is highly likely that it carries out its function via influencing the activity of other PcG members i.e. PRC1 and PRC2.

The invention therefore provides a mouse model for mesothelioma, comprising knock-out alleles of a Cyclin Dependent Kinase Inhibitor 2a gene (Cdkn2a) and a Cyclin Dependent Kinase Inhibitor 2b gene (Cdkn2b), and conditional knockout alleles of Bap1, Nf2 and p19Arf that can be activated at least in the mesothelial lining of the thoracic cavity.

The CDKN2b-CDKN2a locus on chromosome 9p21 in human is frequently lost in cancer. The locus encodes three cell cycle inhibitory proteins: p15^{INK4b} encoded by CDKN2b, p16^{INK4a} encoded by CDKN2a and p14^{ARF}, encoded by an alternative reading frame of CDKN2a (Gil and Peters, 2006. Nature Rev Mol Cell Biol 7: 667-677). Preferred mice are deficient for all three open reading frames (Cdkn2ab^{-/-}). Said mice preferably are generated from transgenic lines in which the mouse homologous sequences of Ink^{4a} and Ink^{4b} are deleted, followed by a recombination to remove the intermediate ARF sequence. Suitable Cdkn2ab^{-/-} mice have been described (Krimpenfort et al., 2007. Nature 448: 943-946).

Said conditional alleles preferably can be activated by a recombination event mediated by a site specific recombinase system such as Cre-LoxP and Flp-FRT systems. Cre recombinase is an enzyme that removes DNA by homologous recombination between binding sequences known as Lox-P sites. The Flip-FRT system operates in a similar way.

Said conditional alleles of Bap1, Nf2 and p19Arf comprise genomic fragments that are flanked by either Lox-P sites or FRT sites. After provision of a recombinase, recombination of these Lox-P sites or FRT sites result in deletion of the genomic fragment that is flanked by either Lox-P sites or FRT sites.

Said recombinase, preferably Cre recombinase, is preferably provided by intrathoracic administration, preferably by viral-mediated intrathoracic administration, more preferably by adenovirus administration.

### EXAMPLES

### Example 1

### Materials and Methods

Generation of Bap1 Conditional Knock Out Mouse (Bap1^{l/f})

ES cells with conditional targeted exons 6-12 of Bap1 were obtained from European conditional Mouse Mutant repository EUCOMM. A Frt-flanked premature stop cassette containing lacZ and neomycin cassette was inserted upstream. Southern blot-verified ES cells were expanded and injected into blastocyst to generate chimera. Chimeras were then bred with BL/6 mice to obtain germline-transmitted mice. Subsequently they were crossed with Flpe mice to remove the lacZ cassette from the Bap1 locus. Bap1^{l/fl}, Bap^{fl/+}, and Bap1^{+/+} littermate mice were genotyped by PCR with the primers Bap1F1 (CTCAATATTCCACCCTGCGTCTG), Bap1R1 (GGCAGGTGGCC CCTCTACTCTA) listed in 5′-3′ order using the following parameters: 95° C. for 5 min, followed by 30 cycles of 94° C. for 30 s, 56° C. for 30 s, and 72° C. for 40 s, and then 72° C. for 5 min. The WT allele was detected at 250 bp, and the floxed allele was detected at 356 bp by PCR.

Nf2 conditional deletion mouse has been described by Giovanini et al. [Giovannini et al., 2000. Genes Dev 14: 1617-30] and Cdkn2ab mice have been described by Krimpenfort et al. [Krimpenfort et al., 2007. Nature 448: 943-6].

All animal work were carried out according to the protocols approved by institutional animal experiments committee (DEC—Dier experimenten commissie) of The Netherlands Cancer Institute, Amsterdam, The Netherlands. Mice were housed under standard feeding, light and temperature with ad libitum access to food and water. All animals used had mixed genetic background.

To obtain mesothelioma cell lines, a small piece of mesothelioma primary tumor was chopped into fine pieces and put in modified HITES medium (DMEM/F12(1:1) GIBCO, supplemented with Glutamax, 4 ug/ml hydrocortisone (Sigma), 5 ng/ml murine EGF (Invitrogen), insulin-transferin-selenium solution (GIBCO), 10% FCS (GIBCO) and penicillin and streptomycin (GIBCO). The chopped tissues were grown in 370c in 5% C02 for 2-3 days. The cells that attached to the culture dish and grew out % were passaged and cell lines were established.

Mesothelioma cells were cultured in DMEM F12 Glutamax supplemented with 10% fetal bovine serum, 4 ug/ml hydrocortisone (Sigma), 5 ng/ml murine EGF (Invitrogen), Insulin-Transferrin-Selenium solution (GIBCO), penicillin and streptomycin (GIBCO).

For the induction of mesothelioma in mouse, six to eight weeks old mice were treated with cyclosporine A (Novartis) in drinking water one week before the adenovirus administration and 2-3 weeks following the infection. The mice were injected intrathoracically with 5×109 cfu purified adenovirus with Cre recombinase driven from the ubiquitous CMV promoter. The injected volume was 50 ul. For injection, the mice were temporarily sedated with Ketamine: Sedazine: Nacl (2:1:17) by injecting 100 ul per 10 gm of mouse weight intraperitoneally. The injection site was cleaned with 70% alcohol and 50 μl virus particles were injected with an insulin injection needle through the side of the rib into the thoracic space and content of the syringe was slowly released.

For histological analysis lungs were inflated with formalin. Other tissue harboring tumors were also fixed with formalin for 24 hours. Fixed tissues were subsequently dehydrated and embedded in paraffin and 2 um sections were cut and stained for hematoxylin and eosin. For immunohistochemistry tissue sections were rehydrated, blocked in BSA containing PBS and subsequently incubated with primary antibodies and subsequently with secondary antibodies.

Immunohistochemistry was carried out for Wilm's tumor protein 1 (Santa Cruz, sc-192), cytokeratin wide spectrum (DakoCytomation, Z0622), podoplanin (Abcam ab11936), F4/80 (AbD Serotec, MCA497). Signaling pathways were examined by immunohistochemistry with following antibodies: p-S6 (Cell Signaling #2211), p-AKT (Cell Signaling #4060), p-ERK (Cell Signaling #4370), and YAP-TAZ (cell signaling #8418).

For gene expression analysis, RNA was isolated from tumor tissue with the Qiagen All prep DNA/RNA kit. Quantification and quality assessment for RNA were performed with a Bioanalyzer (RIN >6.5) (Agilent, Santa Clara, Calif., USA).

Sequencing libraries were constructed with a TruSeq mRNA Library Preparation Kit using poly-A-enriched RNA (Illumina, San Diego, Calif., USA). The samples were run on a HiSeq 2500 Illumina sequencer for cluster generation and sequencing.

For RNA sequencing and expression analysis, Illumina TruSeq mRNA libraries were sequenced with 51 base single reads on a HiSeq2000 using V3 chemistry (Illumina Inc., San Diego). The sequence reads were mapped to the mouse genome (mm10), using TopHat (2.0.12). TopHat was run with default. Reads with mapping quality less than 10 and non-primary alignments were discarded. Remaining reads were counted using HTSeq-cout. Statistical analysis of the differential expression of genes was performed using DESeq2. Genes with False Discovery Rate (FDR) for differential expression lower than 0.05 were considered significant. Batch effect within tumor samples from different source was corrected using ComBat with default options through the Bioconductor sva package 3.10.

For short term colony formation assay, the mesothelioma cell lines were seeded in 500 cells per well in a 384-well plate. The next day drugs (GSK126, AZD4547, AZD2461, ABT-263) were added in a matrix format at the indicated concentration using HP D300 digital dispenser (HP) and cells were grown in presence of drug(s) or DMSO control for 5 days. Thereafter, 10% v/v Alamar blue was added to the well and incubated at 370 C for 4 hours. The plates were read with Tecan reader. The data were analyzed and plotted for drug response curve.

For colony formation assays, the mesothelioma cell lines were seeded in 5,000 cells per well in a 6-well plate and allowed to adhere overnight. The next day GSK126 and/or AZD4547 were added (at the indicated concentration) and cells were grown in presence of drug or DMSO control for 7 days. At the end plates were simultaneously fixed and stained with 6% glutaraldehyde with 0.1% crystal violet solution and digitalized on a image scanner. All experiments were performed at least twice and representative results are shown.

For in vivo treatment of BNC mice, we induced deletion of floxed alleles by injecting Adeno-CMV-Cre in to the pleural space of mice. Six weeks following injection, mice were randomized into treatment and vehicle groups. Tumor induced mice were treated with i. cisplatin and pemetrexed, or vehicle for six weeks: ii. GSK126 and AZD4547, or vehicle for six weeks. Mice were sacrificed when they showed signs of sickness such as consistent weight loss and breathing difficulties. The organs such as thoracic wall, heart, lungs, esophagus, and diaphragm were collected for histology. Tumor burden was scored by a pathologist. A score of 10 represents highest tumor burden, while a score of 0 represents absence of tumor.

### Results

Bap1 Deletion, Together with Nf2, p19Arf, and Cdkn2ab Leads to Rapid Malignant Mesothelioma Development

Recently, the gene encoding BAP1 has been reported to be mutated, lost, or silenced in multiple cancers including mesothelioma [Carbone et al., 2013. Nat Rev Cancer 13: 153-9; Carbone et al., 2012. J Transl Med 10: 179; Pilarski et al., 1993. in GeneReviews®, R. A. Pagon, et al., Eds., Seattle (Wash.); Goldstein, 2011. Nat Genet 43: 925-6; Harbour et al., 2010. Science 330: 1410-3]. We generated a conditional knockout mouse of Bap1 to study the contribution of BAP1 loss to tumorigenesis. Homozygous germline deletion of Bap1 in mice results in early embryonic lethality [Dey et al., 2012. Science 337: 1541-6]. Therefore, Bap1 conditional mice were generated using ES cell clones obtained from EUCOMM and carrying a conditional allele of Bap1. In this conditional allele, exon 6-12 of Bap1 are flanked by loxP sequences. Exon 6-12 encodes part of the ubiquitin carboxyl-terminal hydrolase domain, BARD1 and the HCFC1 interacting domain. Upon Cre mediated recombination, exon 6-12 of Bap1 will be deleted creating a nonfunctional gene in line with the observation that homozygous germline deletion of Bap1 exon 6-12 is embryonic lethal.

It was previously shown that thoracic injection of Ad5-CMV-Cre (adenovirus carrying the Cre recombinase gene driven by the CMV promoter) inducing the deletion of Nf2 and p53 conditional alleles in mesothelial cells cause MM [Jongsma et al., 2008. Cancer Cell 13: 261-71]. Similarly, we deleted the Bap1 allele in thoracic cavity of adult mice by intrathoracic injection of an Ad5-CMV-Cre virus to determine whether Bap1 deletion was sufficient to induce mesothelioma. However, deletion of Bap1 in the thoracic cavity in a cohort of 13 mice did not result in mesothelioma during the lifetime of the mice (mice were monitored for up to 700 days). This implies that Bap1 deletion alone is insufficient to induce mesothelioma. However, we did observe tumors in other tissues such as a hepatoma in one mouse and a lymphoma in another mouse out of 11 homozygous floxed mice 17 months after Ad5-CMV-Cre virus instillation.

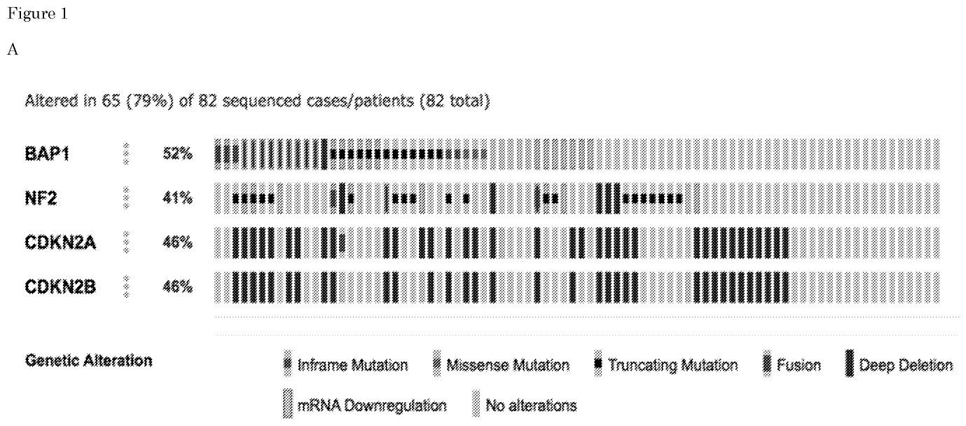

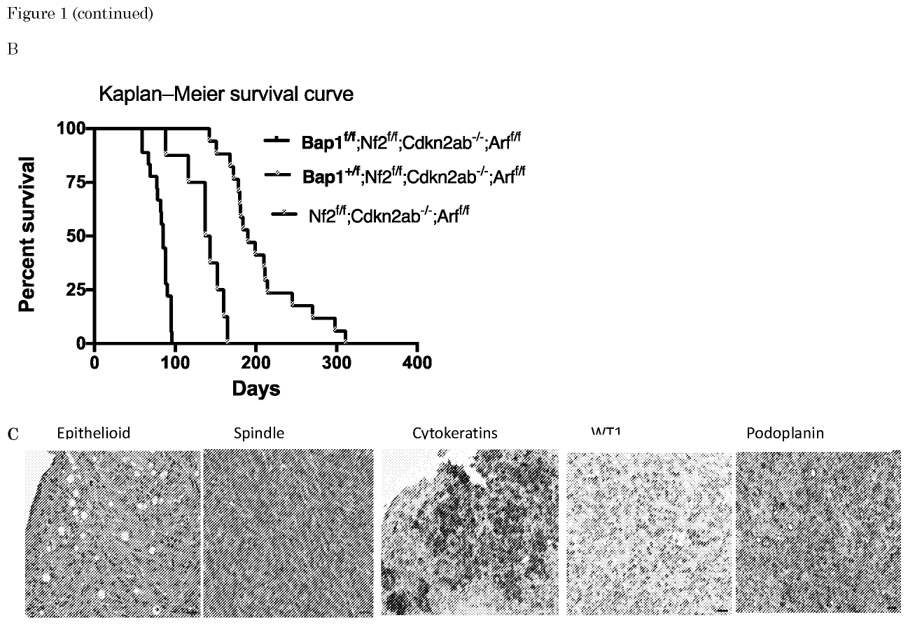

BAP1 alteration is one of the most frequent event in mesothelioma comprising of more than half of the cases of mesothelioma. Along with BAP1 the tumor suppressors NF2 and CDKN2A, CDKN2B are frequently mutated or deleted in mesothelioma as described in TCGA dataset (FIG. 1A). To determine whether deletion of Bap1 along with NF2, CDKN2AB locus deletion would accelerate mesothelioma development, we crossed the conditional Bap1 allele into the Nf2f/f; Cdkn2ab−/− model. The combined loss of Bap1, Nf2, p19Arf and Cdkn2ab (hereafter refer as BNC) in the mesothelial lining of the thoracic cavity by Ad5-CMV-Cre gave rise to mesothelioma in all mice included in the cohort (FIG. 1B). Mice in which Nf2, p19Arf, and Cdkn2ab (hereafter refer as NC) were deleted but with functional Bap1 alleles gave rise to mesothelioma in 75-80% of mice included in the cohort. The median latency of mice homozygously deleted for NC was ˜200 days while the additional loss of Bap1 reduced the latency to less than 70 days. This implies that this combination of lesions is sufficient for rapid and reproducible tumor development.

The tumors that arose in this mouse model were exclusively thoracic malignant mesothelioma. The lesions were mainly composed of epithelioid cells showing nest, nodular and sheet-like arrangements (FIG. 1C). Moreover, the tumor cells were medium to large and round to oval in shape. Additionally, they contained rather rich foamy cytoplasm that converted to large single vacuoles in some of the tumor cells. A small percentage of the tumors also had cells with spindle-shaped appearance and organized in fascicular structures (FIG. 1C). Immunohistochemistry of wide spectrum cytokeratin, Wilms tumor-1, and podoplanin confirmed the mesothelioma characteristics of these lesions (FIG. 1C).

Tumor penetrance is 100% in mice homozygous deleted for all three BNC loci. This also holds for NC mice in combination with one conditional allele of Bap1. Mice with heterozygous deletion of Bap1 showed an intermediate latency that was still substantially faster than NC mice indicative of a dose dependent effect of Bap 1 deletion (FIG. 1B). In the latter case, immunohistochemistry showed that many tumor cells still express the protein indicating incomplete or no loss of heterozygosity (LOH) indicating that the intermediate latency was not caused by the time it took to lose the functional Bap1 allele by LOH but rather that the BAP1 tumor suppressor activity acts in a dose-dependent fashion.

### BNC Deletion Leads to Aggressive Disease and Induce Predominantly Epithelioid Mesothelioma

BAP1 deletion has been reported in many epithelial cancers. Homozygous deletion of Bap1 predominantly induced epithelioid mesothelioma in our mouse model. This is in line with the literature on human MM with homozygous deletions of BAP1 [Yoshikawa et al., 2012. Cancer Sci 103: 868-74]. 3 out of 12 mice presented with biphasic MM that mostly consisted of epithelioid cells with a small fraction of spindle cells. Further confirmation of mesothelioma was obtained by immunohistochemistry with epithelioid markers such as WT-1, keratin wide spectrum, and podoplanin (see FIG. 1C). Since MM is often misdiagnosed and needs to be distinguished from lung adenocarcinoma and rhabdomyosarcoma we performed TTF-1 and MYF-4 antibody staining to rule out lung adenocarcinoma and rhabdomyosarcoma. The absence of staining for TTF-1 and MYF-4 (data not shown) indicates the tumors do not represent adenocarcinoma or rhabdomyosarcoma and therefore represent MM.

The MMs with disrupted Bap1 were highly aggressive and invasive mimicking advanced stage of invasive mesothelioma as observed in man. Macroscopic examination of the thorax demonstrated multiple, irregular, and grayish lesions in esophagus, ribs, and sternum together with invasive growth in muscles, and diaphragm (data not shown).

### BAP1 Deficient Mesothelioma Shows Augmented Activation of P3K and MAPK Pathways

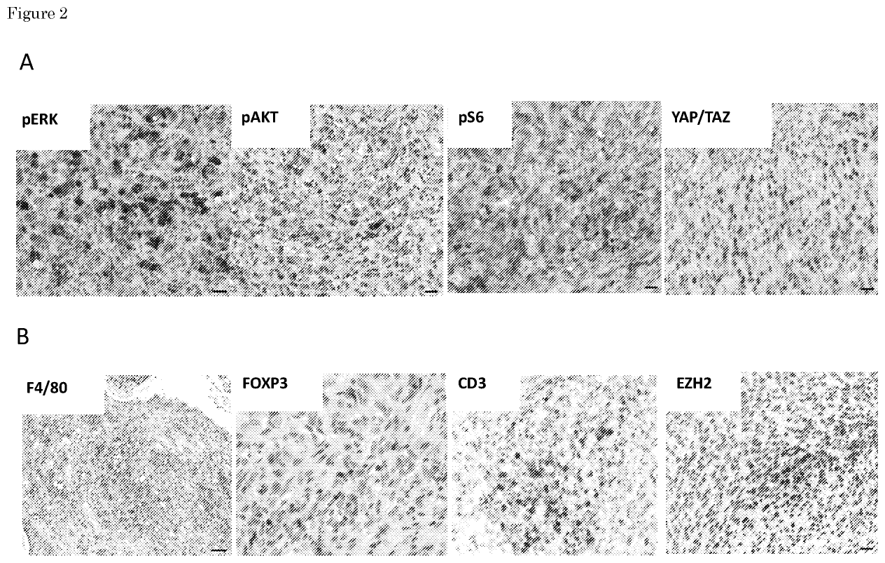

In mesothelioma, multiple oncogenic pathways have been implicated including PI3K, MAPK, and focal adhesion kinase(FAK) pathways. [Ramos-Nino et al., 2005. Mol Cancer Ther 4: 835-42; Shukla et al., 2011. Int J Cancer 129: 1075-86; Marek et al., 2014. Mol Cancer Res 12: 1460-9; Uematsu et al., 2003. Cancer Res 63: 4547-51; Miyanaga et al., 2015. J Thorac Oncol 10: 844-51; Fujii et al., 2012. J Exp Med 209: 479-94; Mizuno et al., 2012. Oncogene 31: 5117-221. Therefore, we explored activation of these pathways by immunostaining. We observed prominent MAPK pathway activation as evidenced by phospho ERK staining (FIG. 2A). PI3K pathways activation is very predominant although patchy reflecting tumor heterogeneity as evident from p-AKT staining (FIG. 2A). The downstream effectors of these pathways such as pS6K also stain positively (FIG. 2A). The Hippo signaling pathway (Pan, 2010. Dev Cell 19: 491-505) is highly active in human mesothelioma as part of downstream merlin NF2 signaling cascade. We observed clear activation of YAP/TAZ in mouse MM (FIG. 2A) [Mizuno et al., 2012. Oncogene 31: 5117-22]. Notably, our mouse models reconfirmed previously reported expression of FAK in mesothelioma [Poulikakos et al., 2006. Oncogene 25: 5960-8; Shapiro et al., 2014. Sci Transl Med 6: 237ra68], further emphasizing the close resemblance of our mouse tumor model with the cognate human disease. PI3K and MAPK pathway activation is already evident in the initial tumor lesions in line with the notion that the combination of engineered lesions is sufficient to activate this pathway and thereby responsible for the swift synchronous tumor onset. In summary, our mouse model closely recapitulates the activation of the signaling pathways as observed in the human mesothelioma.

### BNC Derived Tumors Recapitulate the Immunophenotype of Human Mesothelioma

Inflammation is closely associated with MM and is mostly ascribed to the inflammatory response inflicted by exposure to asbestos fibers [Robinson and Lake, 2005. N Engl J Med 353: 1591-603]. This raises the question whether our mesothelioma model that is exclusively based on engineered tumor suppressor gene deletions would lack this specific aspect of mesothelioma. Therefore, we assessed the presence of macrophages, and tumor infiltrating lymphocytes (TILs) in tumor slices. H&E staining showed presence of infiltrating cells in the tumor compartment. We observed abundant macrophage marker staining (F4/80) in mouse mesothelioma (FIG. 2B). Presence of a substantial number of regulatory T cells was evident from FOXP3 and CD3 staining whereas tumor associated neutrophils were largely absent (FIG. 2B). The strong immunophenotype of the mouse mesothelioma model indicates that this phenotype is dictated by cell-type specific features and might be less dependent on the parameters that are critical for tumor induction (e.g. asbestos exposure, or high mutation load) although these might be important for the success of subsequent interventions.

### Bap1 Loss Drives a Repressive Transcriptional Program

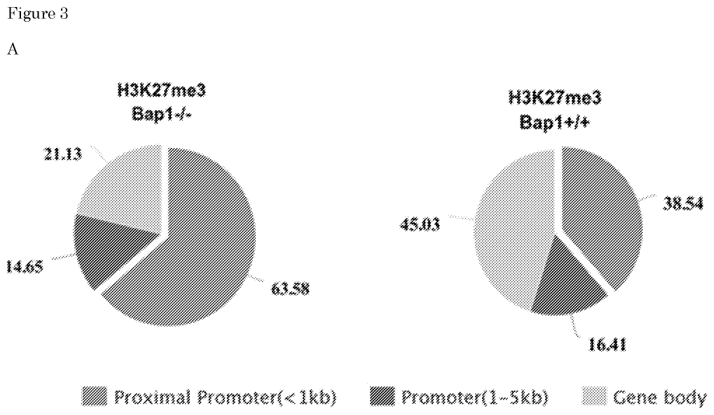

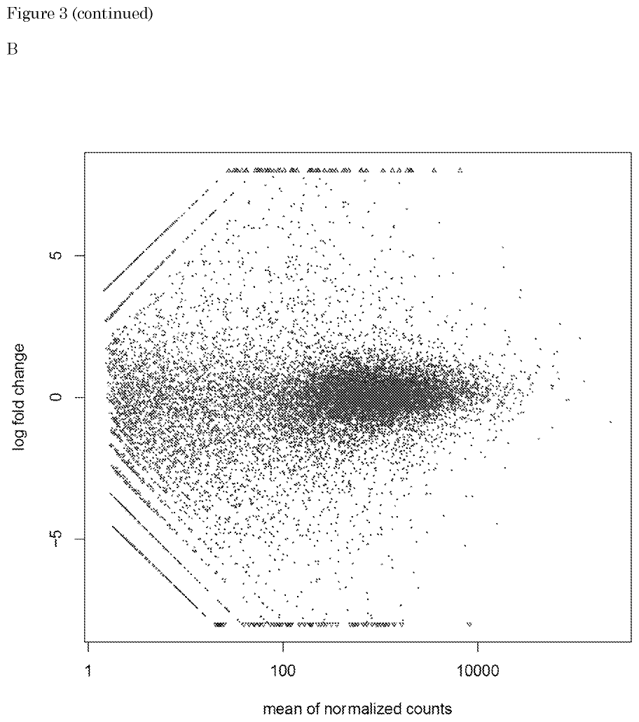

Bap1 loss results in elevated EZH2 expression and increased H3K27 trimethylation. To gain further insight into how Bap1 loss causes an increase in H3K27me3 and promote faster tumor development, we exploited genome-wide chromatin profiling. Analysis of ChIP-seq profiles for H3K27me3 occupancy at gene promoters, CpG islands, and gene bodies revealed a striking pattern of increased methylation around transcriptional start sites. The global promoter sites occupancy of H3K27me3 in BAP1-deficient cells (78%) is significantly higher than in BAP1 proficient cells (27%). Moreover, the binding in gene bodies is significantly lower in BAP1 deficient cells i.e., 18% as compared to 72% in proficient cells (FIG. 3A). The observations point towards redistribution of polycomb repressive marks in Bap1 deleted cells towards promoter sites leading to global downregulation of multiple genes. The increase H3K27me3 at promoter sites can explain the prominent downregulation of the genes found in BAP1-deficient cells. We found 1731 deregulated genes (p<0.01 and >2 fold upregulated or down regulated), of which more than 65% showed decreased expression upon BAP1 loss (FIG. 3B). KEGG pathway enrichment analysis suggests that BAP1 loss does affect multiple cancer related pathways.

### FGFR Inhibition and EZH2 Inhibition is Synergistic in Mesothelioma

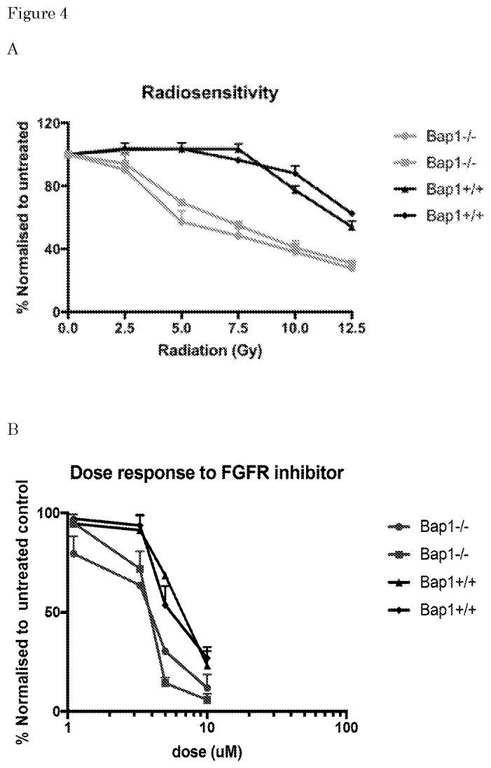

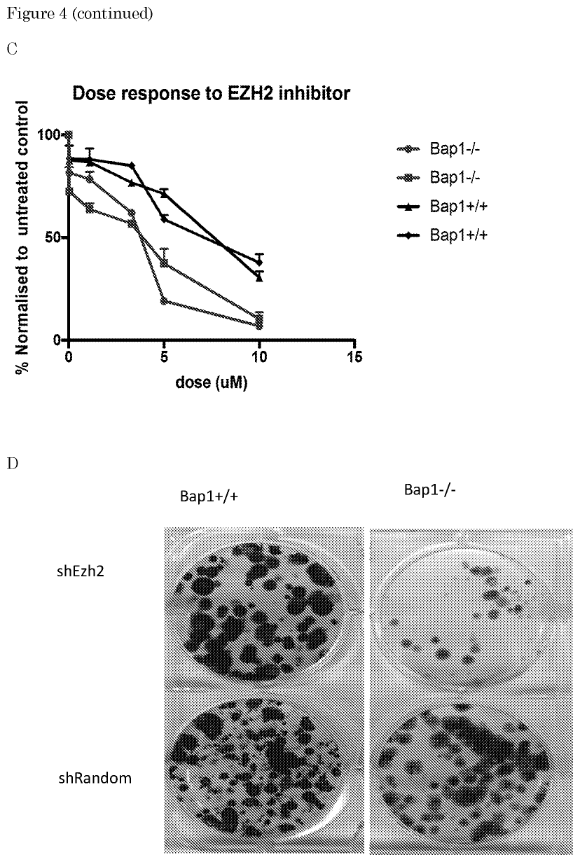

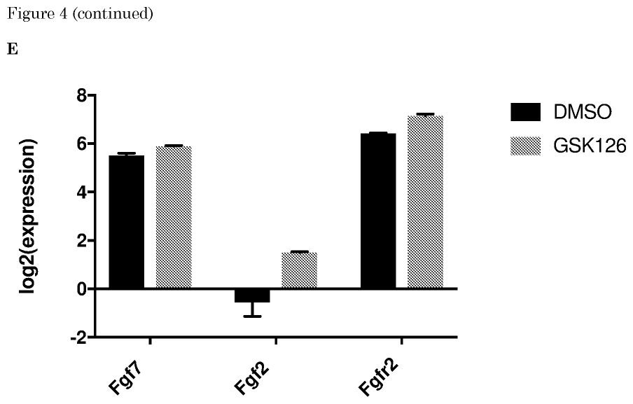

To determine whether BAP1 depletion confers a distinct drug response profile we determined the response of a panel of BAP1-deficient and proficient cell lines to various drugs. The cell lines used here were early passage (within the first 4-8 passage) lines derived from tumors isolated from BNC and NC mice. Bap1 deleted mesothelioma cells appear hypersensitive to radiation (FIG. 4A). We have observed BAP1 deficient cells are hypersensitive to FGFR inhibitor AZD4547 (FIG. 4B) and EZH2 inhibitor GSK126 (FIG. 4C), as was previously found for BAP1 mutated human mesothelioma cell lines (LaFave et al., 2015, Nat Med 21: 1344-1349; Quispel-Janssen et al., 2018, Clin Cancer Res 24: 84-94). This sensitivity was further confirmed by depleting Ezh2 by shRNA knockdown (FIG. 4D). This further confirms that the mouse mesothelioma tumor cells show a drug response profile that closely mimics human MM subtype. We found that EZH2 inhibitor GSK126 treated cells showed upregulation of FGF/FGFR pathway genes, when compared to DMSO treated cells (FIG. 4E). This prompted us to combine EZH2 inhibitor and FGFR inhibitor in mesothelioma. Interestingly, the combination of FGFR inhibitor (AZD4547) and EZH2 inhibitor (GSK126) is synergistic for BAP1 deficient cells. Both the mouse mesothelioma cells and human mesothelioma tested showed synergistic combination (FIG. 5A). We have shown that combining both drugs completely kill all the cells while multiple colonies survive when treated with the single agents (FIG. 5A). We have further demonstrated, the synergy score values for Bap1 negative mouse mesothelioma and human mesothelioma cells are higher than 690, indicating a strong synergy (FIG. 5B) (Prichard, 1990. Antiviral Res 14: 181-206). This indicates FGFR1-3 inhibition together with EZH2 inhibition is synergistic.

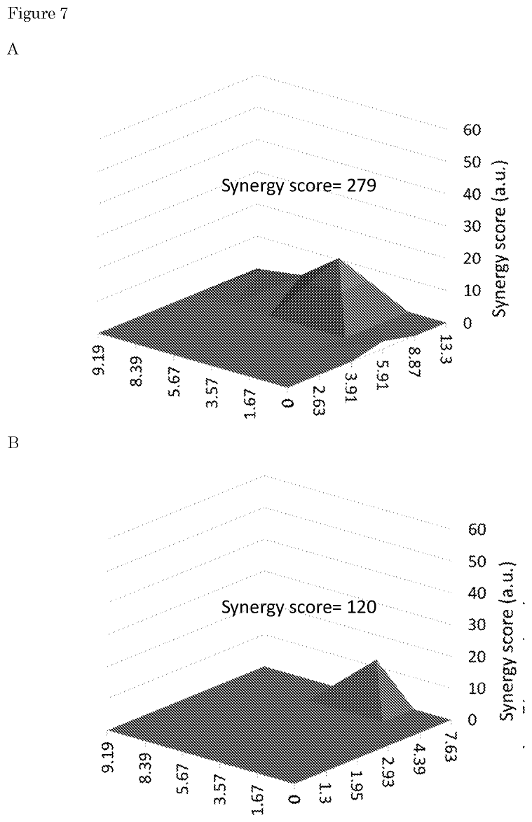

Additionally, we have tested several inhibitors that were reported to be effective as single agent in a mesothelioma drugs screen [Kolluri et al., 2018, eLIFE 7: e30224] in combination with EZH2 inhibitor GSK126. To our surprise, we did not observe strong synergy in 4 such combinations (see FIG. 7). This indicates that even though these drugs were proposed to be effective as single agent, they do not produce a strong synergistic effect in combination with an EZH2 inhibitor. Therefore the combination of EZH2 inhibitor and FGFR inhibitor represents a novel, highly effective combination for treating BAP1-deficient mesothelioma cells.

We have further explored this combination in a BNC autochthonous mouse model of mesothelioma. Mesothelioma tumor was induced in BNC mice by injecting adeno-Cre in the thoracic cavity. Six weeks following this injection, mice were treated with vehicle (DMSO) or with a combination of GSK126 and AZD4547 for 6 weeks. Mice treated with a combination of GSK126 and AZD4547 had significantly reduced thoracic tumor burden, when compared to vehicle treated mice (FIG. 5C).

### BNC Autochthonous Models Allows Preclinical Testing of Treatment Modalities

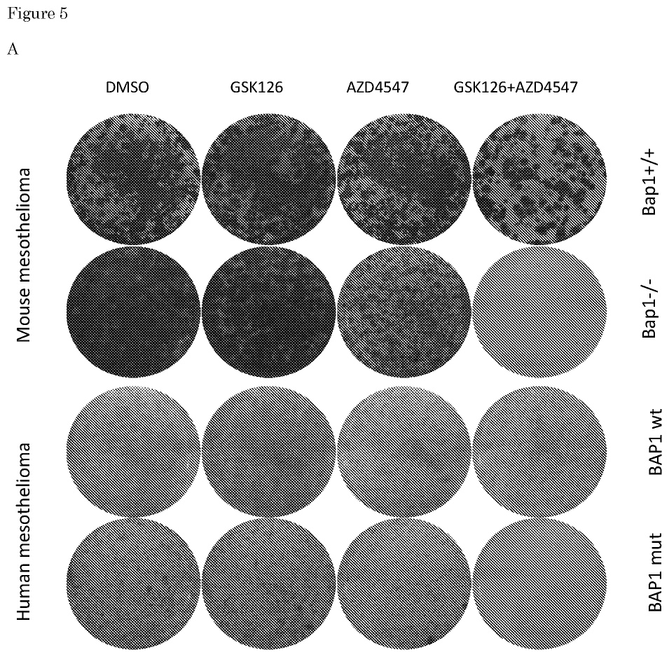

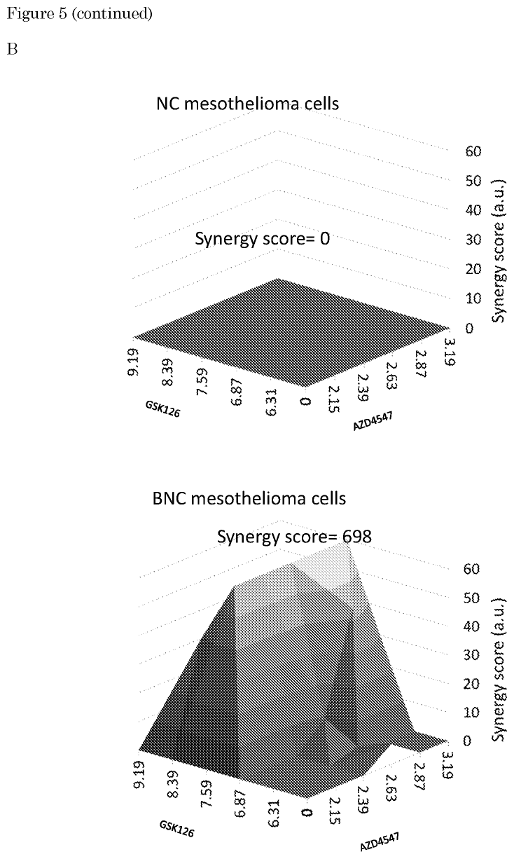

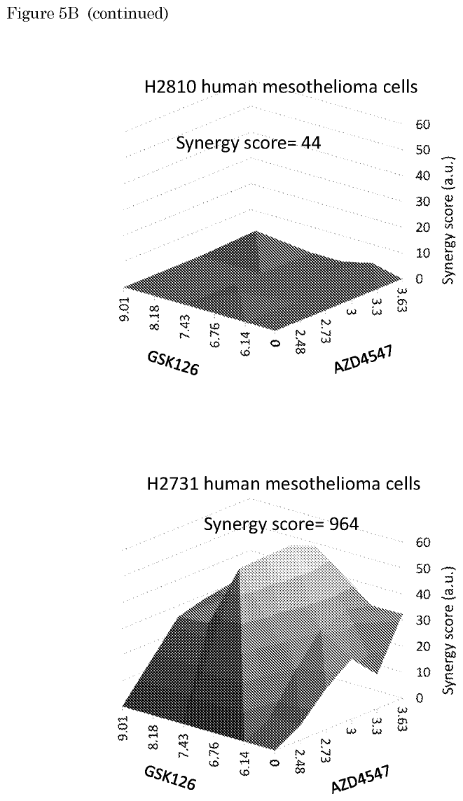

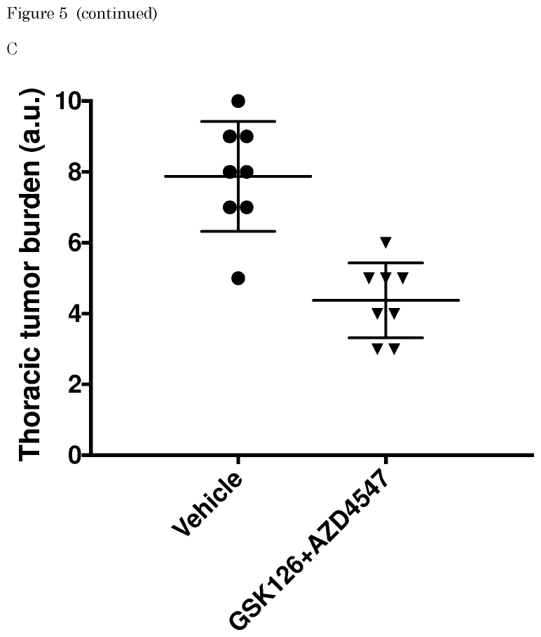

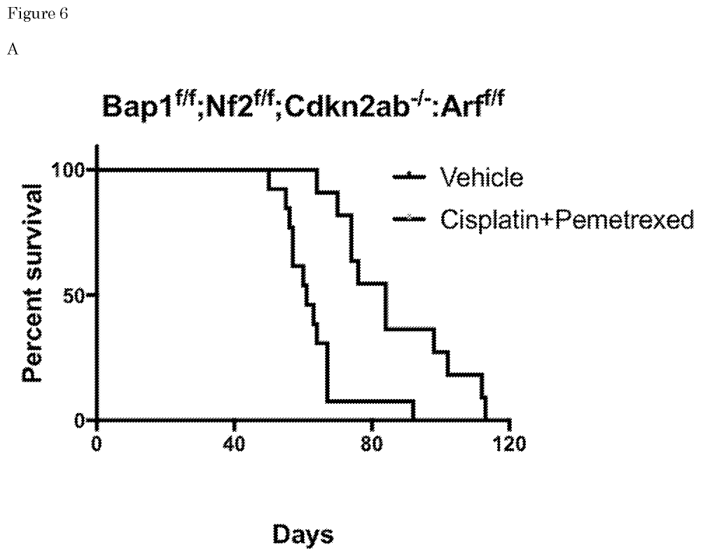

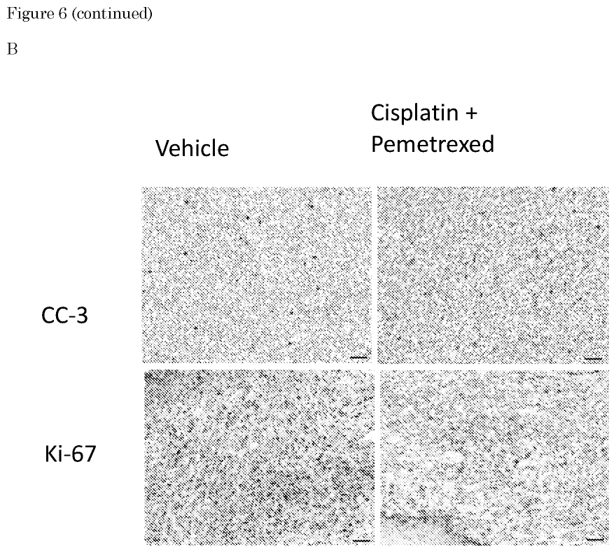

The current frontline therapy for mesothelioma patients is cisplatin plus pemetrexed. We tested how BNC responds to this treatment. Therefore, we induced the deletion of floxed alleles by Ad-CMV-Cre injection and started administration of cisplatin and pemetrexed 6 weeks later. The treatment of cisplatin and pemetrexed prolonged survival of the mice with approximately 3 weeks (FIG. 6A). Treated tumors exhibited a significant increase in p53 activation and cleaved caspase in IHC (FIG. 6B). This illustrates that this autochthonous mouse model allows accurate assessment of drug regimens and is well-suited as an intervention model without requiring tumor graft strategies. The model is fast and specific and has narrow window of tumor development.

1. A method of treating a patient suffering from a BRCA1-associated protein 1 (BAP1) negative cancer, the method comprising providing the patient with a combination of a FGFR inhibitor and an EZH2 inhibitor.

2. The method according to claim 1, wherein said FGFR inhibitor is a selective FGFR1, FGFR2, and/or FGFR3 inhibitor.

3. The method according to claim 1, wherein said FGFR inhibitor is AZD4547.

4. The method according to claim 1, wherein said EZH2 inhibitor is EPZ-6438, GSK126, or a combination thereof.

5. The method according to claim 1, wherein said patient has previously been treated with one or more chemotherapeutical agents, in combination with either pemetrexed or raltitrexed.

6. The method according to claim 1, wherein said patient has become resistant to said one or more chemotherapeutical agents.

7. The method according to claim 1, wherein said method comprises providing the combination to the patient in cycles of 2-6 weeks, with at least one day of rest in between the cycles.

8. The combination for use according to claim 7, wherein the number of cycles is 4-10.

9. The combination for use according to claim 1, wherein the method of treatment is combined or alternated with a G2 checkpoint abrogator, a focal adhesion kinase inhibitor, gemcitabine, or a combination thereof.

10. A pharmaceutical preparation, comprising a FGFR inhibitor and an EZH2 inhibitor.

11. The pharmaceutical preparation according to claim 10, comprising a pharmaceutical preparation comprising a FGFR inhibitor and a pharmaceutical preparation comprising an EZH2 inhibitor.

12. The pharmaceutical preparation according to claim 10, for use in a method of treating BRCA1-associated protein 1 (BAP1) negative cancer.

13. A method of identifying a patient with cancer, especially mesothelioma, eligible for treatment with a combination of a FGFR inhibitor and an EZH2 inhibitor, comprising testing a biological sample comprising cancer cells from the patient for the presence of a mutation in BAP1 and/or loss of BAP1, wherein the patient is eligible for treatment with said combination if said tumor cells in said sample test positive for a mutation in BAP1.

14. The method of claim 13, wherein said step of testing a biological sample from the patient for the presence of a mutation in BAP1 and/or loss of BAP1 comprises:
(a) providing a bodily fluid from the patient,
(b) magnetically separating cancer cells from essentially all other cells in said sample using magnetic nanoparticles comprising antibodies that target cancer cells, and
(c) determining the presence of a mutation in BAP1 and/or loss of BAP1 in cancer cells in said cancer cell-enriched sample, preferably by immunohistochemistry.

15. A mouse model for mesothelioma, comprising knock-out alleles of Cdkn2a and Cdkn2b, and conditional knockout alleles of Bap1, Nf2 and p19Arf that can be activated at least in the mesothelial lining of the thoracic cavity.

16. The method of claim 1, wherein the BRCA1-associated protein 1 (BAP1) negative cancer is a BAP1-negative mesothelioma.

17. The method of claim 4, wherein said EZH2 inhibitor is GSK126.

18. The method of claim 5, wherein said patient has previously been treated with cisplatin or carboplatin.

19. The pharmaceutical composition of claim 12, wherein the BRCA1-associated protein 1 (BAP1) negative cancer is a BAP1-negative mesothelioma, especially BAP1-negative mesothelioma.
